(19) United States
(12) Patent Application Publication
(10) Pub. No.: US 2023/0088180 A1
(43) Pub. Date: Mar. 23, 2023

(54) MOBILE DEVICE AS POWER METER

(71) Applicant: Apple Inc., Cupertino, CA (US)

(72) Inventors: Stefan Meyer, Hoechstadt (DE); Norman Goris, Dortmund (DE)

(21) Appl. No.: 17/482,248

(22) Filed: Sep. 22, 2021

(51) Int. Cl. B62J 45/415; G01L 3/10; B62J 45/20; G01C 22/00; H04W 4/80

(52) U.S. Cl. CPC B62J 45/415; G01L 3/10; B62J 45/20; G01C 22/002; H04W 4/80

(57) ABSTRACT

Aspects of the subject technology relate to communication device used as a power meter. The communication device includes circuitry to determine values of several forces and a processor. The processor determines a combined force by combining the determined values of the forces. The processor further determines a value of a power based on the s combined force, a speed and a loss factor. The communication device is used by a user to measure the power. The power is generated by the user when engaged in an activity, and the forces affect a movement of the user.

### TECHNICAL FIELD

The present description relates generally to handheld electronic devices, and more particularly, but not exclusively, to using a mobile device as a power meter.

### BACKGROUND

Bicycling is done all around the world. Typically, the value of a parameter called functional threshold power (FTP) is used to judge a rider's fitness level. This value is measured in watts per kilogram and can be acquired via a predefined ride while constantly measuring the power produced by the rider. The FTP value is then used to define certain training ranges for the rider. At a certain ambition level, the rider constantly monitors the produced power level and adjusts the gear or speed to work in an optimal range. Together with heart rate, cadence, speed and blood oxygen level, the power measurement can be the perfect match for biking. It can also be used and extended to other types of long(er)-distance sports such as marathon running or walking.

Currently, power meters for biking are implemented in special pedals or axes to be mounted on the bike. The devices then need to be connected to a small global positioning system (GPS) device or a speed meter. The devices collects the data and displays it to the rider. Typical costs for two pedals or a dedicated axis is in the order of 1,200-2,000 Euros. Also, for running, there are sensors that have to be added to the runner's shoes to enable measuring the power. Thus, the existing solutions are quite expensive and deliver only limited accuracy and can only be used for a single application (e.g., bike or shoes). In addition to the very expensive power meters, there are devices available that can perform a crude power estimation based on the speed and weight of a rider.

### BRIEF DESCRIPTION OF THE DRAWINGS

Certain features of the subject technology are set forth in the appended claims. However, for purposes of explanation, several aspects of the subject technology are set forth in the following figures.

FIG. 1 is a block diagram illustrating an example system for using a portable communication device as a power meter, in accordance with various aspects of the subject technology.

FIG. 2 is a flow diagram illustrating an example algorithm for using a portable communication device as a power meter, in accordance with various aspects of the subject technology.

FIG. 3 is a flow diagram illustrating an example algorithm for static and dynamic calibration and adjustment of power measurement by a portable communication device, in accordance with various aspects of the subject technology.

FIG. 4 is a schematic diagram illustrating an example of a neural network used for machine learning-based determination of power-calculation parameters, in accordance with various aspects of the subject technology.

FIG. 5 illustrates a wireless communication device within which some aspects of the subject technology are implemented.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be clear and apparent to those skilled in the art that the subject technology is not limited to the specific details set forth herein and may be practiced without these specific details. In some instances, well-known structures and components are shown in block-diagram form in order to avoid obscuring the concepts of the subject technology.

The subject disclosure is directed to using a portable communication device as a power meter. Examples of the portable communication device include a smartphone, a smartwatch, a tablet or any other portable communication device. The power meter of the subject technology can measure the power generated by a user of the portable communication device when engaged in a sport such as riding a bike, walking, running, swimming or other similar sports. However, most examples discussed in this disclosure may refer to bicycling as an example sport. Here as an example, the FTP value is calculated as watts per kilogram (W/Kg) and can be determined via a predefined ride while constantly measuring the power produced by the rider.

The existing power meters are either quite expensive or can only perform a crude power estimation based on the speed and weight of a rider. For example, expensive biking power meters are implemented in special pedals or axes to be mounted on the bike and need to be connected to a small global positioning system (GPS) device or speed meter for collecting and displaying data. The pedals and even the axis need to be especially tailored for fitting to the bike they are attached to, which makes them unusable for other kinds of bikes. Even if the power meter fits to several bikes the interchanging is far from easy. The axis removal requires a complete built up of the bike, while the constant attaching and reattaching of the pedals limits the lifetime of the power meter. Further, the existing power meters designed for cycling are not usable for other sports such as running or walking.

The techniques of the subject technology can achieve an accurate power measurement using a user's portable communication device without a need for additional equipment or a computer, which makes the disclosed technique inexpensive and simple to use.

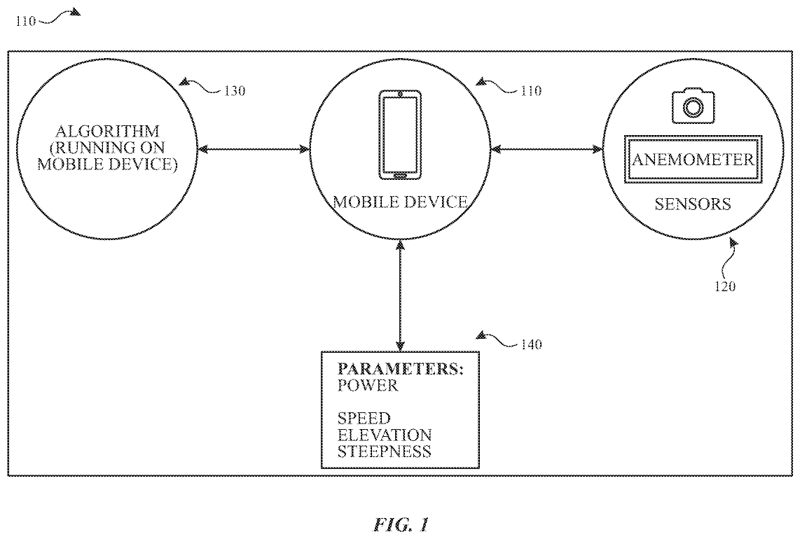

FIG. 1 is a block diagram illustrating an example system 100 for using a portable communication device as a power meter, in accordance with various aspects of the subject technology. The system 100 includes a portable communication device 110 (e.g., a smartphone or a smartwatch, hereinafter, device 110), a set of sensors 120 and an algorithm 130, and several parameters 140. In some implementations, the sensors 120 may include a camera and an anemometer, which may be the camera of the device 110. The algorithm 130, in some implementations, can run on the portable communication device 110. Examples of the parameters 140 include the generated power, a position, a speed, an elevation, the steepness of a path, a height, a wind speed, a rolling resistance and so on. The subject technology combines a set of sensors 120 with the device 110 and the algorithm 130 to compute capabilities for building a mobile power meter. Accordingly, the power meter of the subject technology is inexpensive and can be interchangeable between applications, users and different types of sports. In other words, the power meter of the subject technology solves the problems of the currently available solutions. Combined with the typical capabilities of the device 110, such as the GPS, temperature measurements and gyro, a perfect combination for covering all relevant parameters for a variety of sports can be provided.

The anemometer measures air speed, which together with the rider speed measurement on the device 110 (e.g., via a GPS) can be used to calculate the additional power required by the rider to compensate for each headwind. The camera is used to obtain information about the type and quality of the path. This is another important measure that defines the required power. The camera is just one way to detect the street quality. Another way would be to use the GPS information and associated map information, which may not cover details such as wet paths. The sensors 120 can continuously send sensor data to the device 110 for computation of the generated power. The parameters 140 can also include weight of the rider and weight of the bike, which are preconfigured. The position of the rider is a relevant parameter for power calculation, for example, it makes a difference whether the driver is riding low or high. The rider position can be measured via a gyro sensor and movement sensors on the device 110.

The rolling resistance parameter can be constructed based on several other parameters, such as the path type, quality and condition, which can be detected by the camera system, and wheel type and width, which can be preconfigured. The wind speed is measured by the anemometer, and the ride speed can be measured by the GPS of the device 110 or by a speed sensor connected to the bike. The path slope or steepness can be provided by the device 110 including a steepness sensor, which typically relies on air pressure, and its calibration can be done via a GPS device. The height parameter is relevant to air density and can be deducted via a barometric height formula from the ambient pressure and temperature. The power measurement is performed over time and an average power is calculated over a preconfigured period.

The values of the above-specified parameters can be used to create the power values for the current ride. Furthermore, a complete setup of data relevant to the rider can be displayed, averaged and postprocessed to allow for efficient training and riding. Thus, the device 110 can not only replace a power meter but also the overall bike computer. Furthermore, the other sensors 120 of the device 110, such as heart rate and blood oxygen measurement sensors, can be used to deliver an even more complete experience. Also, parameters such as a bike geometry or a user height can be beneficial to increase the accuracy for biker position impacts on the power values.

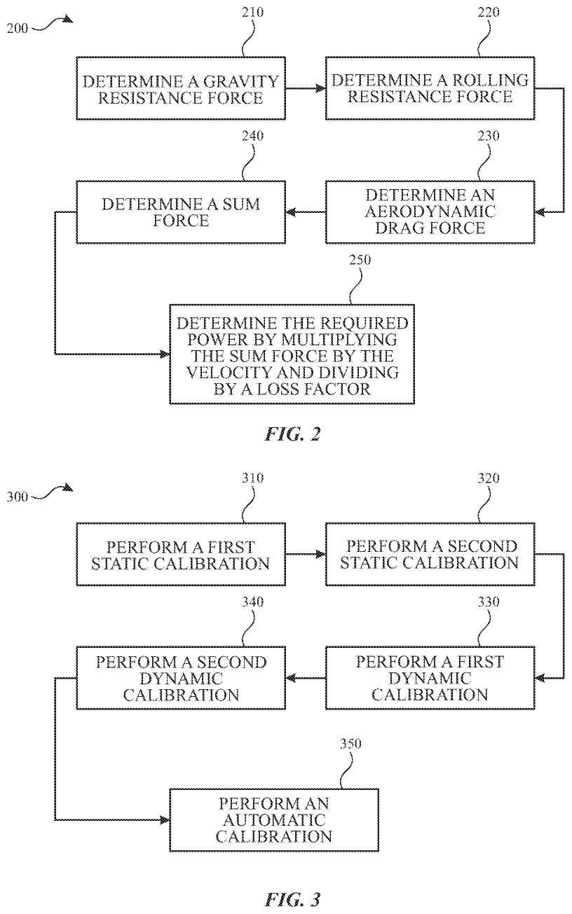

FIG. 2 is a flow diagram illustrating an example algorithm 200 for using a portable communication device as a power meter, in accordance with various aspects of the subject technology. The algorithm 200 starts at operation block 210, where a gravitational resistance force (F_{g}) is determined. The gravitational resistance is the force induced when the user is riding uphill or downhill and it depends on the gravitational constant g, the slope, as well as the combined mass (m+M) of the bike and the rider and is expressed as:

F_{g}=g*sin(arc tan(slope))*(m+M) (1)

At operation block 220, a rolling resistance force (Fᵣ) is determined. The rolling resistance force is used to overcome the rolling resistance and depends on the slope as well as the combined mass (m+M) of the bike and rider. In additional implementations, a rolling resistance factor is added, which depends on the surface type as well as the type of tires. For the rolling resistance factor (Cᵣᵣ) value, a set of different semi-empirical tables exist. Typical ranges are between 0.0001 and 0.05. In the context of this application these tables are only used as a guideline, and machine learning together with sensor input is used to further interpolate and improve these values. The rolling resistance force can be expressed as:

Fᵣ=g*sin(arc tan(slope))*(m+M)*Cᵣᵣ (2)

At operation block 230, an aerodynamic drag force (Fₐ) is determined. The aerodynamic drag force is the force to overcome the aerodynamic resistance. It mainly depends on the frontal area (A) as well as the relative speed raised to the second power and a set of constants. In addition, air density (ρ) is included, which depends on the height (h) and temperature (T). The aerodynamic drag force can be expressed as:

Fₐ=0.5*(C_{d}*A)*ρ(h, T)*(V_{own}−V_{wind})² (3)

The term (Cd*A) typically depends on the position of the rider and is defined semi-empirically, and it is typically within a range of 0.2 and 0.5. In the following description, the semi-empirical table will just be used as a starting point complemented by additional machine learning as well as sensor input. The relative speed (V_{own}−V_{wind}) term represents the riding speed (V_{own}) relative to the wind speed (V_{wind}).

At operation block 240, a summation of the three forces discussed above (F_{g}+Fᵣ+Fₐ) is calculated. At operation block 250, the required power (P) is determined by using the following expression:

P=(F_{g}+Fᵣ+Fₐ)*V/(1−loss) (4)

V is the speed in m/s and loss represents the loss due to additional sources in percentage. Additional loss due to chain and wheel ball bearings and bottom brackets of the bike can be within the range of about 1.5% to 3%, depending on the condition of the bike.

Some of the parameters used in the above expressions are preconfigured, for example, the weight of the bike and rider. Other parameters can be taken from a set of preconfigured tables. However, these tables are only used as a baseline and are further extended by machine learning using a neural network, as discussed below.

The rolling resistance factor (Cᵣᵣ) is defined by the type of tire as well as the type of path. The other relevant parameters such as a camera input, a gyro device input and an ultrawideband (UWB) input are inputs for the machine learning and/or artificial intelligence to estimate the path resistance coefficient. Typically, a front camera and a rear camera are available at the device 110 of FIG. 1 (e.g., a smartphone). While the rear camera is used to check the path, the front camera can be used to check the position of the driver. Note that in order to save power, the camera is only used when the gyro sensor detects a motion of the driver. The input from the gyro device of distance and orientation of the smartwatch or smartphone is used to track relative motion of the rider. Further, a relative position of the smartphone (attached to the bike) and the smartwatch can be measured using a UWB input, which can also be used to deduce the change in the rider's position as well as the long-distance position (aerodynamic position). In order to derive these values, additional information such as the height of the rider and the geometry of the bike can be beneficial to enhancing accuracy.

The combined value of drag coefficient (Cd) the times frontal area (A) is typically defined by the position of the driver. Parameters such as camera input, gyro input and UWB input are used and input for the machine learning/artificial intelligence to estimate the road resistance coefficient. Typically, a front and rear camera is available at the phone. While the rear camera is used to check the road, the front camera is used to check the position of the driver. Note that in order to save power the camera is only used when the gyro sensor detects a motion of the driver. Relative motion of the user can be tracked via the gyro in the watch or the phone (depending on the attachment position of the device). Distance between the smartphone and, for example, the smartwatch can be used to define the relative distance and orientation to yards each other. This value can also be used to deduce the change of the biker position as well as the position itself. In order to derive these values, the height of the driver as well as the geometry of the bike are also beneficial to increase the accuracy. All the above parameters need to be trained and incorporated into the coefficient calculation algorithm together with a set of pre-defined tables.

There can be various scenarios for attaching the phone or other devices. In one scenario, a smartwatch and a smartphone (mounted on the handlebars) can be used. This scenario is the typical deployment as a bike computer displaying speed and other parameters. All sensors of the phone can be used as well as the front and rear cameras. The associated watch used on the wrist can be used to deliver complementary data on the driver position, gyro, as well as distance to the phone and other devices to the bike. In another scenario, the smartwatch and the smartphone can be used where the user puts the smartphone in her/his shirt. In this scenario, the smartphone at the back of the shirt allows a more reliable tracking of the bike position. While the camera cannot be used, the other sensors such as a lidar or a UWB device are still available. In a third scenario, only the phone is used, similar to the two other scenarios discussed above, however, interaction with the watch would be missing. The fourth scenario involves using the smartwatch only, which of course results in further reduced sensor input and the least accuracy. In order to deliver the best accuracy, the combination of the smartwatch and the smartphone in the first and the second scenario is recommended.

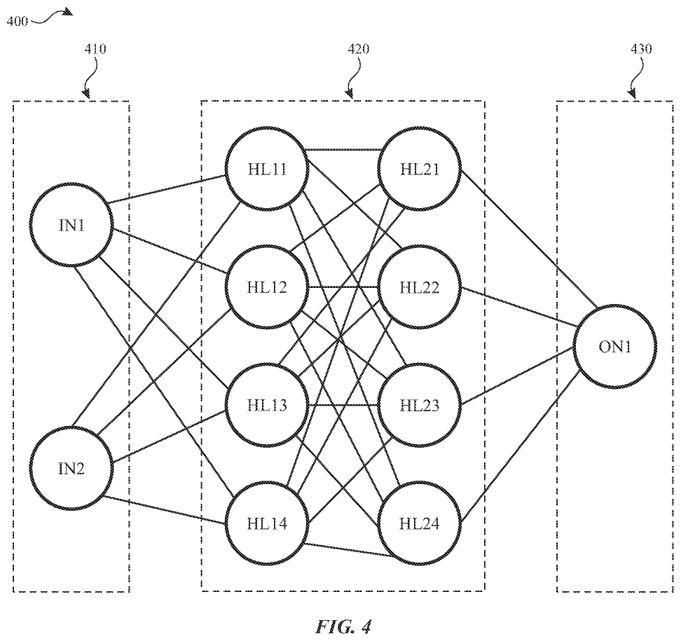

FIG. 3 is a flow diagram illustrating an example algorithm 300 for static and dynamic calibration and adjustment of power measurement by a portable communication device, in accordance with various aspects of the subject technology. The algorithm 300 begins at operation block 310, where a first static calibration is performed. In the first static calibration, the rider accelerates to a speed of about 10 km/h and rolls until the speed reaches about 5 km/h. This would allow deriving rolling resistance base values, as the wind pressure is low.

At operation block 320 a second static calibration is performed. In the second static calibration, the rider accelerates to a speed of about 35 km/h and rolls until the speed reaches about 25 km/h. This makes the wind pressure dominate, and an associated rolling resistance vale can be derived. Both first and second static calibrations should be performed in the same rider position. A higher accuracy can be achieved by repeating the first and second static calibrations in other rider positions.

At operation block 330 a first dynamic calibration is performed. In the first dynamic calibration, the rider accelerates to a speed of about 10 km/h and holds the speed for about 30 seconds. This would allow to calibrate the Cᵣᵣ value.

At operation block 340 a second dynamic calibration is performed. In the second dynamic calibration, the rider accelerates to a speed of about 30 km/h and holds the speed for about 30 seconds. This would allow to calibrate the Cᵣᵣ values for the wind pressure. Both first and second dynamic calibrations should be performed in the same rider position. A higher accuracy can be achieved by repeating the first and second dynamic calibrations in other rider positions.

At operation block 350 an automatic calibration and adjustment is performed. For the automatic calibration and adjustment, typically rolling parts of the trip are used. Here the gyro device is used to detect that the rider is currently not pedaling and thus the applied power is zero. Depending on the speed and other parameters, the speed gradient can be used to deduce and adjust the parameters relevant for the wind pressure and the rolling resistance. Another way to trigger automatic calibration and adjustment is the measurement of position changes. Assuming that a constant pedal force for a short period around the position changes the speed decrease/increase can be used to further enhance the user-specific values for wind and path resistance force.

A similar concept can be used when a path change is detected. Assuming an almost constant power for a short period around the change in the speed increase and/or decrease can be used to further refine the parameters for the power calculation. The dynamic calibration and adjustment also allows for accounting for dynamic changes that cannot be directly detected via sensors, e.g., weight loss due to sweating.

FIG. 4 is a schematic diagram illustrating an example of a neural network 400 used for machine learning-based determination of power-calculation parameters, in accordance with various aspects of the subject technology. The neural network 400 has an input layer 410, a hidden layer 420 and an output layer 430. The input layer 410 includes, but is not limited to, two input nodes IN (IN1 and IN2), where the input values for training the neural network 400 are applied. Accordingly, the number of nodes of the input layer 410 depends on the number of input parameters used for training. The hidden layer 420 includes, but is not limited to, neurons HL (HL11, HL12, HL13, HL14, HL21, HL22, HL23, HL24). Each input node IN is connected to all neurons of the first hidden layer HL1 (HL11, HL12, HL13, HL14) and each neuron of the first hidden layer HL1 is connected to all neurons of the second hidden layer HL2 (HL21, HL22, HL23, HL24). Further, all neurons of the second hidden layer HL2 are connected to an output node (ON1) of the output layer 430. In some implementations, the output layer 430 can have more than one output node, each of which would be connected to all neurons of the second hidden layer HL2. During the training process, the weights of the connections between neurons of the hidden layer are adjusted to produce one or more desired output. Once the training is finished, the final values of the weights of the connections is used to define an output for any given set of input vales.

In one or more implementations, the subject technology can use a neural network, such as the neural network 400, to determine a number of parameters that cannot be preconfigured. For example, the parameters such as Cᵣᵣ and C_{d}*A can be determined using a neural network. The parameter Cᵣᵣ, for instance, is determined based on inputs from the camera, motion sensor, GPS, maps and weather condition, as discussed above. A series of such inputs can be applied to a neural network (e.g., 400) to train the neural network to be able to produce the parameter Cᵣᵣ for any future values of the inputs. Also, the drag coefficient (C_{d})*frontal areas (A), which is a function of the position of the rider, as described above, is dependent on inputs from camera input, the gyro device and the UWB device. These inputs can be used to train the neural network 400 or a similar neural network in order to determine values of the C_{d}*A for a desired future situation.

The path condition is another parameter to be used for training the neural network. For example, a motion sensor can be used to detect the bumpiness of the path which, along with the path type information, can be incorporated into the path condition approximation. For example, the higher the bumpiness of the path, the higher the path resistance. Also, GPS and maps can be employed to set some kind of base value for the path condition to be used as a starting point for the other sensor inputs and updated. The other inputs can be, for example, the current weather condition that can be obtained from an online application (app). The current weather condition such as rain, snow and so on can add an input layer to the coefficient calculation.

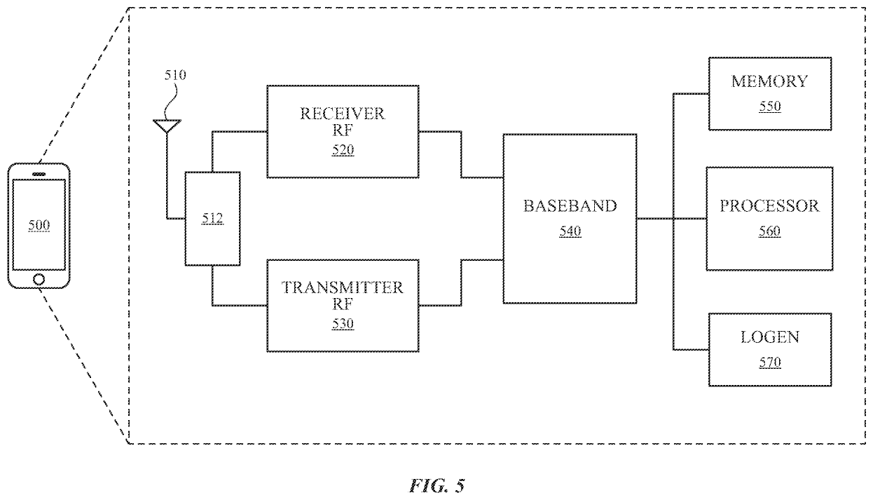

FIG. 5 illustrates a wireless communication device within which some aspects of the subject technology are implemented. In one or more implementations, the wireless communication device 500 can be a smartphone, a smartwatch, a tablet or other electronic devices that may be used to implement the power meter of the subject technology. For example, the wireless communication device 500 can be the device 110 of FIG. 1. The wireless communication device 500 may comprise a radio-frequency (RF) antenna 510, a duplexer 512, a receiver 520, a transmitter 530, a baseband processing module 540, a memory 550, a processor 560 and a local oscillator generator (LOGEN) 570. In various aspects of the subject technology, one or more of the blocks represented in FIG. 5 may be integrated on one or more semiconductor substrates. For example, the blocks 520-570 may be realized in a single chip or a single system on a chip or may be realized in a multichip chipset. The wireless communication device 500 can also include a camera, a UWB device, a gyro and a GPS device that can be used to implement some aspect of the subject technology as described above.

The receiver 520 may comprise suitable logic circuitry and/or code that may be operable to receive and process signals from the RF antenna 510. The receiver 520 may, for example, be operable to amplify and/or downconvert received wireless signals. In various aspects of the subject technology, the receiver 520 may be operable to cancel noise in received signals and may be linear over a wide range of frequencies. In this manner, the receiver 520 may be suitable for receiving signals in accordance with a variety of wireless standards such as Wi-Fi, WiMAX, Bluetooth, and various cellular standards. In various aspects of the subject technology, the receiver 520 may not use any sawtooth acoustic wave (SAW) filters and few or no off-chip discrete components such as large capacitors and inductors.

The transmitter 530 may comprise suitable logic circuitry and/or code that may be operable to process and transmit signals from the RF antenna 510. The transmitter 530 may, for example, be operable to upconvert baseband signals to RF signals and amplify RF signals. In various aspects of the subject technology, the transmitter 530 may be operable to upconvert and amplify baseband signals processed in accordance with a variety of wireless standards. Examples of such standards may include Wi-Fi, WiMAX, Bluetooth, and various cellular standards. In various aspects of the subject technology, the transmitter 530 may be operable to provide signals for further amplification by one or more power amplifiers.

The duplexer 512 may provide isolation in the transmit band to avoid saturation of the receiver 520 or damaging parts of the receiver 520, and to relax one or more design requirements of the receiver 520. Furthermore, the duplexer 512 may attenuate the noise in the receive band. The duplexer 512 may be operable in multiple frequency bands of various wireless standards.

The baseband processing module 540 may comprise suitable logic, circuitry, interfaces, and/or code that may be operable to perform the processing of baseband signals. The baseband processing module 540 may, for example, analyze received signals and generate control and/or feedback signals for configuring various components of the wireless communication device 500, such as the receiver 520. The baseband processing module 540 may be operable to encode, decode, transcode, modulate, demodulate, encrypt, decrypt, scramble, descramble, and/or otherwise process data in accordance with one or more wireless standards.

The processor 560 may comprise suitable logic, circuitry, and/or code that may enable processing data and/or controlling operations of the wireless communication device 500. In this regard, the processor 560 may be enabled to provide control signals to various other portions of the wireless communication device 500. The processor 560 may also control transfer of data between various portions of the wireless communication device 500. Additionally, the processor 560 may enable implementation of an operating system or otherwise execute code to manage operations of the wireless communication device 500. In one or more implementations, the processor 560 can be used to execute algorithm 300 of FIG. 3. The processor 560 can also be used to implement the neural network 400 of FIG. 4.

The memory 550 may comprise suitable logic, circuitry, and/or code that may enable storage of various types of information such as received data, generated data, code, and/or configuration information. The memory 550 may comprise, for example, RAM, ROM, flash, and/or magnetic storage. In various aspects of the subject technology, information stored in the memory 550 may be utilized for configuring the receiver 520 and/or the baseband processing module 540. In some implementations, the memory 550 may store data (coordinates) of the 3-D point map of selected points of the torso of the user of the subject technology.

The LOGEN 570 may comprise suitable logic, circuitry, interfaces, and/or code that may be operable to generate one or more oscillating signals of one or more frequencies. The LOGEN 570 may be operable to generate digital and/or analog signals. In this manner, the LOGEN 570 may be operable to generate one or more clock signals and/or sinusoidal signals. Characteristics of the oscillating signals such as the frequency and duty cycle may be determined based on one or more control signals from, for example, the processor 560 and/or the baseband processing module 540.

In operation, the processor 560 may configure the various components of the wireless communication device 500 based on a wireless standard according to which it is designed to receive signals. Wireless signals may be received via the RF antenna 510, amplified, and downconverted by the receiver 520. The baseband processing module 540 may perform noise estimation and/or noise cancellation, decoding, and/or demodulation of the baseband signals. In this manner, information in the received signal may be recovered and utilized appropriately. For example, the information may be audio and/or video to be presented to a user of the wireless communication device 500, data to be stored to the memory 550, and/or information affecting and/or enabling operation of the wireless communication device 500. The baseband processing module 540 may modulate, encode, and perform other processing on audio, video, and/or control signals to be transmitted by the transmitter 530 in accordance with various wireless standards.

It is well understood that the use of personalty identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

Various functions described above can be implemented in digital electronic circuitry, as well as in computer software, firmware or hardware. The techniques can be implemented by using one or more computer program products. Programmable processors and computers can be included in or packaged as mobile devices. The processes and logic flows can be performed by one or more programmable processors and by one or more programmable logic circuitries. General and special-purpose computing devices and storage devices can be interconnected through communication networks.

Some implementations include electronic components such as microprocessors and storage and memory that store computer program instructions in a machine-readable or computer-readable medium (alternatively referred to as computer-readable storage media, machine-readable media, or machine-readable storage media). Some examples of such computer-readable media include RAM, ROM or flash memory. The computer-readable media can store a computer program that is executable by at least one processing unit and include sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multicore processors that execute software, some implementations are performed by one or more integrated circuits such as application-specific integrated circuits or field-programmable gate arrays. In some implementations, such integrated circuits execute instructions that are stored on the circuit itself.

As used in this specification and any claims of this application, the terms “computer,” “processor,” and “memory” all refer to electronic or other technological devices. These terms exclude people or groups of people. For the purposes of the specification, the terms “display” or “displaying” mean displaying on an electronic device. As used in this specification and any claims of this application, the terms “computer-readable medium” and “computer-readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device as described herein for displaying information to the user and a keyboard and a pointing device, such as a mouse or a trackball, by which the user can provide input to the computer. Other kinds of devices can be used to provide for interaction with a user as well, for example, feedback provided to the user can be any form of sensory feedback such as visual feedback, auditory feedback, or tactile feedback. Input from the user can be received in any form, including acoustic, speech, or tactile input.

Many of the above-described features and applications are implemented as software processes that are specified as a set of instructions recorded on a computer-readable storage medium (also referred to as a computer-readable medium). When these instructions are executed by one or more processing unit(s) (e.g., one or more processors, cores of processors, or other processing units), they cause the processing unit(s) to perform the actions indicated in the instructions. Examples of computer-readable media include, but are not limited to, flash drives, RAM chips, hard drives and EPROMs. The computer-readable media does not include carrier waves and electronic signals passing wirelessly or over wired connections.

In this specification, the term “software” is meant to include firmware residing in read-only memory or applications stored in magnetic storage, which can be read into memory for processing by a processor. Also, in some implementations, multiple software aspects of the subject disclosure can be implemented as subparts of a larger program while remaining distinct software aspects of the subject disclosure. In some implementations, multiple software aspects can also be implemented as separate programs. Finally, any combination of separate programs that together implement a software aspect described herein is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language including compiled or interpreted languages and declarative or procedural languages, and it can be deployed in any form including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated tiles (e.g., files that store one or more modules, subprograms, or portions of code). A computer program can be deployed to be executed on one computer or on multiple computers that are located at one site or distributed across multiple sites and interconnected by a communication network.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Some of the blocks may be performed simultaneously. For example, in certain circumstances multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its), and vice versa. Headings and subheadings, if any, are used for convenience only and do not limit the subject disclosure.

The predicate words “configured to,” “operable to,” and “programmed to” do not imply any particular tangible or intangible modification of a subject, but rather are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects, and vice versa. A phrase such as a “configuration” does not imply that such configuration is essential to the subject technology or that such configuration applies to all configurations of the subject technology. A disclosure relating to a configuration may apply to all configurations, or one or more configurations. A phrase such as a configuration may refer to one or more configurations, and vice versa.

The word “example” is used herein to mean “serving as an example or illustration.” Any aspect or design described herein as an “example” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for,” or, in the case of a method claim, the element is recited using the phrase “step for.” Furthermore, to the extent that the terms “include,” “have,” or the like are used in the description or the claims, such terms are intended to be inclusive in a manner similar to the term “comprise,” as “comprise” is interpreted when employed as a transitional word in a claim.

What is claimed is:

1. A communication device used as a power meter, the communication device comprising:
circuitry configured to determine values of a plurality of forces; and
a processor configured to:
determine a combined force by combining the plurality of determined values of the plurality of forces; and
determine a value of a power based on the combined force, a speed and a loss factor,
wherein the communication device is used by a user to measure the power, the power is generated by the user when engaged in an activity, and the plurality of forces affect a movement of the user.

2. The communication device of claim 1, wherein the activity includes cycling, walking, running, and swimming.

3. The communication device of claim 1, wherein the plurality of forces comprises a gravity resistance force, a rolling resistance force and an aerodynamic drag force.

4. The communication device of claim 3, wherein the processor is configured to determine the gravity resistance force induced when riding uphill or downhill based on a path slope parameter measured using a steepness sensor of the communication device.

5. The communication device of claim 3, wherein the processor is configured to determine the rolling resistance force based on a path slope parameter using a steepness sensor of the communication device and a rolling resistance factor, and wherein the processor is further configured to determine the rolling resistance factor using inputs from at least some of a camera, a motion sensor, a global-positioning system (GPS) of the communication device and a neural network implemented by the processor of the communication device.

6. The communication device of claim 3, further comprising determining the aerodynamic drag force based on a frontal area and a relative speed of the user with respect to a wind speed, and wherein the frontal area is determined using inputs from a camera of the communication device and a neural network implemented by the processor of the communication device.

7. The communication device of claim 6, further comprising measuring the wind speed using an anemometer of the communication device.

8. The communication device of claim 3, wherein the speed comprises a user speed, and wherein the processor is configured to measure the user speed using a GPS of the communication device.

9. A power metering system, the system comprising:
a portable communication device including one or more sensors and a processor,
wherein the one or more sensors and the processor are configured to determine a power generated by a user, when the user is engaged in an activity, by estimating values of a plurality of forces that affect a movement of the user; and providing inputs by the one or more sensors for determining additional parameters for determining the power.

10. The system of claim 9, wherein the processor is configured to estimate values of a gravity resistance force, a rolling resistance force and an aerodynamic drag force.

11. The system of claim 10, wherein the processor is configured to estimate a value of the gravity resistance force induced when riding uphill or downhill based on a path slope parameter measured using a steepness sensor of the portable communication device.

12. The system of claim 10, wherein the processor is configured to estimate a value of the rolling resistance force based on a path slope parameter measured using a steepness sensor of the communication device and a rolling resistance factor, and wherein the processor is further configured to estimate the value of the rolling resistance using inputs from at least some of a camera, a motion sensor, a global-positioning system (GPS) of the portable communication device and a neural network implemented by the processor.

13. The system of claim 10, wherein the processor is configured to estimate a value of the aerodynamic drag force based on a value of a frontal area and a relative speed of the user with respect to a wind speed, and wherein the processor is configured to estimate the frontal area using inputs from a camera of the portable communication device and a neural network implemented by the processor.

14. The system of claim 13, wherein the processor is configured to estimate the wind speed using an anemometer of the portable communication device.

15. The system of claim 9, wherein the additional parameters include a rolling resistance factor, a path condition, a drag coefficient, a frontal area, a position of the user.

16. The system of claim 9, wherein the additional parameters are estimated by a neural network trained using inputs from a camera, the one or more sensors including a global-positioning system (GPS), a gyro, an anemometer, an ultrawideband (UWB) sensor and a motion sensor, and online applications operating on the portable communication device.

17. A system for calibrating and adjusting a power meter of a communication device, the system comprising:
memory configured to store power meter data associated with a user-initiated calibration including a first static calibration and a second static calibration; and
a processor configured to perform an automatic calibration and receive a first set of data from one or more sensors while the first static calibration is performed by accelerating to a first speed and rolling until a second speed is reached and to receive a second set of data from the one or more sensors while the second static calibration is performed by accelerating to a third speed and rolling until a fourth speed is reached,
wherein the first static calibration and the second static calibration are performed at a same position of the user, and the memory, the processor and the one or more sensors are implemented in the communication device.

18. The system of claim 17, wherein the processor is configured to derive a rolling resistance base values associated with a first wind speed leveraging the first static calibration.

19. The system of claim 18, wherein the processor is configured to derive the rolling resistance base values associated with a second wind speed leveraging the second static calibration, wherein the second wind speed is higher than the first wind speed.

20. The system of claim 17, wherein the processor is configured to perform two dynamic calibrations while the user is accelerating to the first speed and holding for about 30 seconds and accelerating to the second speed and holding for about 30 seconds, wherein the first speed is about 10 km/hr, the second speed is about 5 km/hr, the third speed is about 35 km/hr and the fourth speed is about 25 km/hr.
